(19) United States
(12) Patent Application Publication
(10) Pub. No.: US 2021/0344886 A1
(43) Pub. Date: Nov. 4, 2021

(54) MEDICAL IMAGE OUTPUT CONTROL DEVICE, RECORDING MEDIUM, MEDICAL IMAGE DISPLAY SYSTEM, AND MEDICAL IMAGE DISPLAY METHOD

(71) Applicant: KONICA MINOLTA, INC., Tokyo (JP)

(72) Inventor: Takafumi NANJO, Tokyo (JP)

(73) Assignee: KONICA MINOLTA, INC., Tokyo (JP)

(21) Appl. No.: 17/238,350

(22) Filed: Apr. 23, 2021

(30) Foreign Application Priority Data
Apr. 30, 2020 (JP) 2020-080015

(51) Int. Cl. H04N 9/79; G16H 30/20; G16H 30/40

(52) U.S. Cl. CPC H04N 9/7908; G16H 30/40; G16H 30/20

(57) ABSTRACT

A medical image output control device that outputs multiple medical images, the device including a hardware processor that controls not to display supplementary information attached to a medical image among the medical images in displaying of a moving image based on the medical images.

### CROSS-REFERENCE TO RELATED APPLICATIONS

The entire disclosure of Japanese Patent Application No. 2020-080015 filed on Apr. 30, 2020 is incorporated herein by reference in its entirety.

### BACKGROUND

### Technological Field

The present invention relates to a medical image output control device, a recording medium, a medical image display system, and a medical image display method.

### Description of the Related Art

There have been conventionally known techniques of attaching supplementary information to medical images and deleting the supplementary information attached to the medical images later by devices that display medical images of still images.

When the medical images are displayed, the attached supplementary information is displayed together with the medical images in the form of annotations indicating the positions of region of interest (ROI) focused on by doctors, captions and the like, for example.

In recent years, supplementary information has been also attached to medical images of moving images (for example, see JP 2018-187310 A).

### SUMMARY

In a case where the medical images are frame images forming a moving image, such supplementary information is possibly attached to only a part of the multiple consecutive frame images. When the moving image having a part of the frame images accompanied with the supplementary information is replayed, the supplementary information flickers on the image (supplementary information is displayed for only a moment). This flickering interferes with interpretation of radiogram by a doctor or the like, depending on circumstances.

An object of the present invention is to avoid flickering of supplementary information when a moving image is replayed.

To achieve at least one of the abovementioned objects, according to an aspect of the present invention, there is provided a medical image output control device that outputs multiple medical images, the device including a hardware processor that controls not to display supplementary information attached to a medical image among the medical images in displaying of a moving image based on the medical images.

To achieve at least one of the abovementioned objects, according to another aspect of the present invention, there is provided a non-transitory recording medium storing a computer readable program that causes a computer of a medical image output control device which outputs multiple medical images to perform moving image output control processing of controlling not to display supplementary information attached to a medical image among the medical images in displaying of a moving image based on the medical images.

To achieve at least one of the abovementioned objects, according to another aspect of the present invention, there is provided a medical image display system that displays multiple medical images, the system including: an image generator that generates the medical images; a display that displays a moving image based on the medical images; and a hardware processor that controls not to display supplementary information attached to a medical image among the medical images in displaying of the moving image by the display.

To achieve at least one of the abovementioned objects, according to another aspect of the present invention, there is provided a medical image display method using a medical image output control device that displays multiple medical images, the method including controlling, by a hardware processor included in the medical image output control device, not to display supplementary information attached to a medical image among the medical images in displaying of a moving image based on the medical images.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a block diagram showing an example of a medical image display system according to an embodiment of the present invention;

FIG. 2 is a block diagram showing another example of a medical image display system according to the embodiment of the present invention;

FIG. 3 is a block diagram showing a medical image output control device included in the medical image display system in FIG. 1;

FIG. 4 is a block diagram showing a medical image output control device included in the medical image display system in FIG. 2;

FIG. 5 is a view showing an example of a display screen displaying the medical image display system in FIG. 3 or 4;

FIG. 6 is a flowchart showing the flow of moving image output control processing executed by the medical image output control device in FIG. 3 or 4; and

FIG. 7 is a view showing multiple medical images displayed as a moving image by each of a conventional device that displays medical images and the medical image, display system in FIG. 1 or 2.

### DETAILED DESCRIPTION OF EMBODIMENTS

Hereinafter, one or more embodiments of the present invention will be described with reference to the drawings. However, the scope of the invention is not limited to the disclosed embodiments or the illustrated examples.

### 1. MEDICAL IMAGE DISPLAY SYSTEM

First, examples of a schematic configuration of a medical image display system according to the present embodiment will be described.

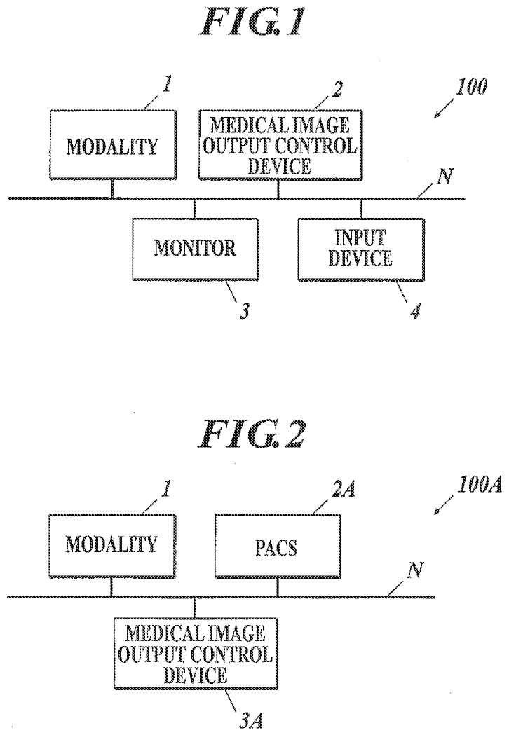

FIG. 1 is a block diagram showing an example of the medical image display system, and FIG. 2 is a block diagram showing another example of the medical image display system.

### 1-1. System Configuration Example 1

As shown in FIG. 1, a medical image display system 100 (hereinafter, referred to as a system 100) according to a configuration example 1 of the present embodiment includes a modality 1, a medical image output control device 2 (hereinafter, referred to as an output control device 2), a monitor 3, and an input device 4.

These components can communicate with each other via a communication network N.

The system 100 may be connected to a hospital information system (HIS), a radiology information system (RIS), an image analyzing device, and the like not shown in the drawings.

### Modality

The modality 1 captures a diagnosis target site in a subject, and generates digital data (hereinafter, image data) of the image (medical image) capturing the diagnosis target site.

The modality 1 includes, for example, an FPD (Flat Panel Detector), a CT (Computer Tomography) device, an MRI (Magnetic Resonance Imaging) device, an ultrasound diagnostic device, or the like.

The modality 1 according to the present embodiment can generate medical images of a moving image by moving image capturing, in addition to medical images of still images by still image capturing.

The above “moving image” indicates an image capable of making a viewer recognize that something appearing in the moving image continuously move by sequentially displaying multiple still images at a high speed.

The moving image capturing in the present invention includes dynamic imaging, and the moving image includes a dynamic image.

The dynamic imaging is obtaining multiple images showing a dynamic state of a subject by repeatedly emitting pulsed radiation such as X ray to a subject at a predetermined time interval (pulse irradiation), or by continuously emitting the radiation to a subject at a low dose rate without interruption (continuous irradiation).

The dynamic image indicates a series of images obtained by the dynamic imaging. Each of the multiple images forming the dynamic image is referred to as a frame image. In such a dynamic image, it is possible to grasp temporal changes in various dynamic structures (for example, moving structures such as lungs and heart) in a human body.

The modality 1 is based on a DICOM standard, and configured to generate image data in a DICOM file format.

The image data in the DICOM file format includes an image portion and a header portion.

The actual data of a medical image is written in the image portion.

The various types of supplementary information are written in the header portion.

The supplementary information is information that is attached to the medical image. That is, the modality 1 is configured to attach the supplementary information to the medical image.

The supplementary information according to the present embodiment includes patient information, test information, series information, image information, GPS (Grayscale Softcopy Presentation State) information, overlay information, and the like.

The patient information includes, for example, the patient ID, patient name, birth date, age, sex, height, and weight.

The test information includes, for example, the test ID, test date, test lime, test description, number of series, and number of images.

The series information includes, for example, the modality, image capturing site, image capturing method (for example, image capturing direction (slice direction), whether contrast medium is used or not), series description, laterality, series number, number of images, and AE title.

The image information includes, for example, the image number, patient direction, image type, pixel structure, vertical width, horizontal width, tube voltage, mA, tube current, pixel interval, slice thickness, slice position, frame number, number of frames, TR, TE, and WW/WC.

When the medical image is a moving image, many modalities including the modality 1 according to the present embodiment generally attach the supplementary information to all the frame images. However, the modality may attach the supplementary information to only a part of the frame images.

The modality 1 according to the present embodiment may be connected to a console (not shown in the drawings) for setting various image capturing conditions and controlling the operation of the modality 1.

The modality 1 may be installed in the image capturing room or configured to be movable.

### Medical Image Output Control Device

The output control device 2 is configured by including a PC, a dedicated device, a virtual server on a cloud, or the like.

The output control device 2 can accumulate the image data which was generated by the modality 1.

The output control device 2 includes the function of a Picture Archiving and Communication Systems (PACS), and stores multiple pieces of image data.

The output control device 2 according to the present embodiment stores multiple pieces of image data in the form of database.

The output control device 2 can output multiple medical images.

The output control device 2 according to the present embodiment performs outputting by transmitting an image signal to an after-mentioned monitor 3.

The details of this output control device 2 will be described later.

### Monitor

The monitor 3 is configured by including an LCD (Liquid Crystal Display), an ELD (Electronic Luminescent Display), or a CRT (Cathode Ray Tube), for example.

The monitor 3 displays a medical image or the like corresponding to the image signal which was received from the output control device 2.

### Input Device

The input device 4 is configured to enable user operations by a keyboard including a cursor key, numeric input keys, and various function keys, a pointing device such as a mouse, a touch panel superposed on the surface of the monitor 3, and the like.

The input device 4 outputs the control signal corresponding to the user operation to the output control device 2.

### Flow of Diagnosis Using Medical Image Display System

The diagnosis using the system 100 according to the present embodiment which is configured in such a way is performed in the following flow.

First, after the user (technologist or the like) performs moving image capturing or still image capturing of a diagnosis target site of a subject by using the modality 1, the modality 1 generates the medical image (moving image or still image) capturing the diagnosis target site.

After the image data is generated, the modality 1 transmits the image data to the output control device 2.

The output control device 2 accumulates the image data which was received from the modality 1.

The output control device 2 performs display control of the image data which was requested by the user (doctor or the like) among the accumulated pieces of image data, and transmits the image signal of the target medical image to the monitor 3.

When the transmitted medical image is a moving image, moving image output control processing (to be described later in detail) is executed in the above display control.

The monitor 3 displays the medical image corresponding to the image signal received from the output control device 2.

The doctor performs diagnosis of the subject on the basis of the medical image displayed on the monitor 3.

### 1-2. System Configuration Example 2

As shown in FIG. 2, a system 100A according to a configuration example 2 of the present embodiment includes a modality 1 similar to that of the above system 100, a Picture Archiving and Communication Systems 2A (hereinafter, referred to as PACS 2A), and a medical image output control device 3A (hereinafter, referred to as an output control device 3A).

These components can communicate with each other via a communication network N.

### PACS

The PACS 2A is obtained by omitting the display control function of medical image from the output control device 2 of the above system 100.

### Medical Image Output Control Device

The output control device 3A is obtained by combining the monitor 3 and the input device 4 of the above system 100 and adding the display control function of medical image.

### Flow of Diagnosis Using Medical Image Display System

The diagnosis using the system 100A according to the present embodiment which is configured in such a way is performed in the following flow.

First, after the user (technologist or the like) performs moving image capturing or still image capturing of a diagnosis target site of a subject by using the modality 1, the modality 1 generates the medical image (moving image or still image) capturing the diagnosis target site.

When the image data is generated, the modality 1 transmits the image data to the PACS 2A.

The PACS 2A accumulates the image data which was received from the modality 1.

The PACS 2A transmits the image data requested by the user (doctor or the like) among the accumulated pieces of image data to the output control device 3A.

The output control device 3A performs display control of the image data received from the PACS 2A and displays the target medical image.

When the transmitted medical image is a moving image, moving image output control processing (to be described later in detail) is executed in the above display control.

The doctor performs diagnosis of the subject on the basis of the medical image displayed by the output control device 3A.

### 2. DETAILS OF MEDICAL IMAGE OUTPUT CONTROL DEVICE

Next, the specific configurations and operations of the output control device 2, 3A included in the above system 100, 100A will be described.

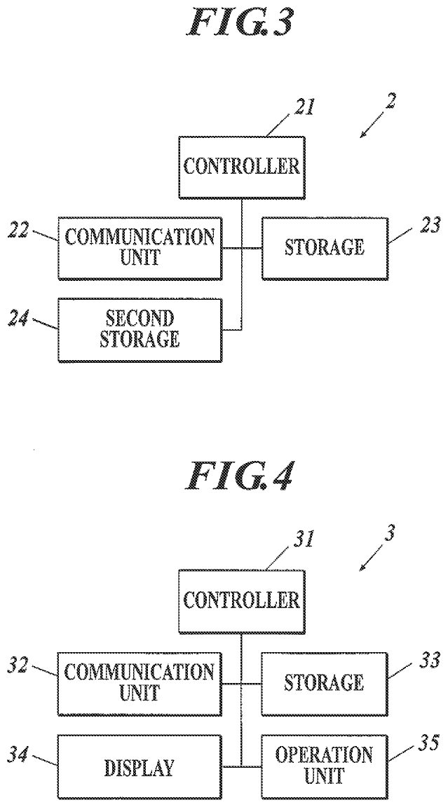

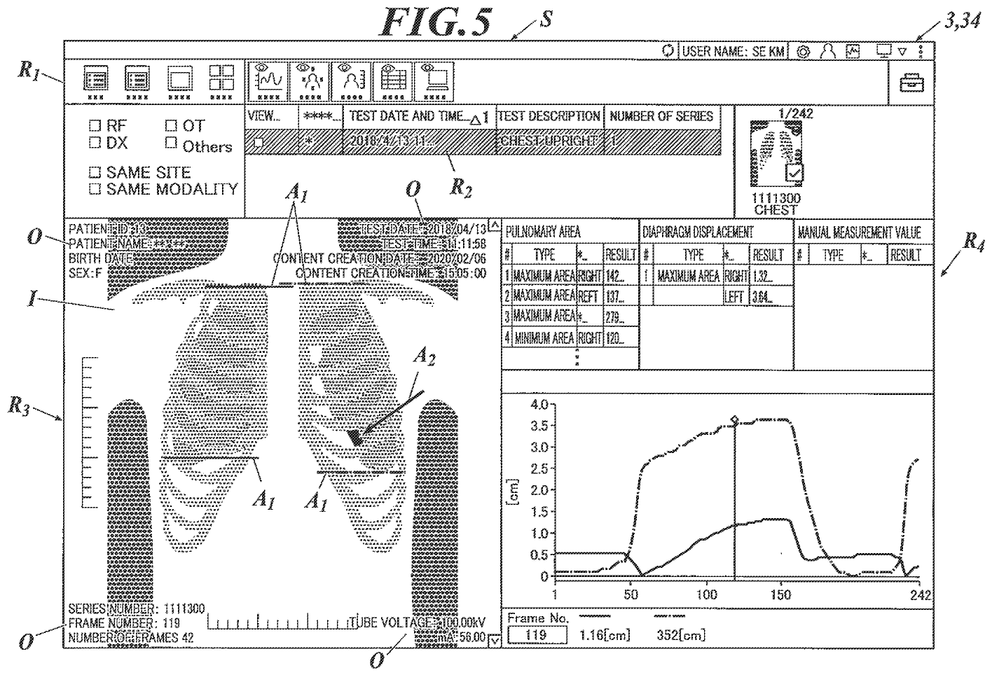

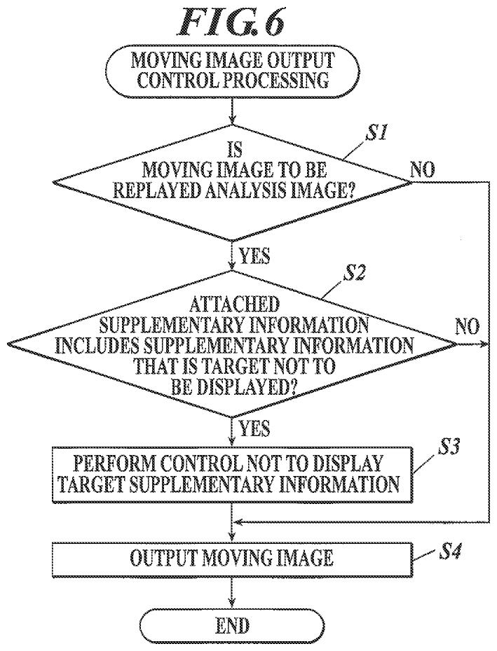

FIG. 3 is a block diagram showing an output control device 2 included in the above system 100. FIG. 4 is a block diagram showing the output control device 3A included in the above system 100A. FIG. 5 is a view showing an example of a display screen displaying the system 100, 100A. FIG. 6 is a flowchart showing the flow of moving image output control processing executed by the output control device 2, 3A.

### 2-1. Configuration Example 1

As shown in FIG. 3, the output control device 2 included in the system 100 according to the above configuration example 1 includes a controller 21 (hardware processor), a communication unit 22, a storage 23, and a second storage 24.

The components 21 to 24 are electrically connected via a bus or the like.

The controller 21 is configured by including a CPU (Central Processing Unit), a RAM (Random Access Memory), and the like.

The CPU of the controller 21 reads out various programs stored in the storage 23 to load them into the RAM, executes various types of processing in accordance with the loaded programs, and integrally controls the operations of the respective components of the output control device 2.

The communication unit 22 is configured by including a communication module or the like.

The communication unit 22 transmits and receives various signals and various types of data to and from other devises (modality 1 and the like) connected via a communication network N (LAN (Local Area Network), WAN (Wide Area Network), internet, or the like).

The storage 23 is configured by including a non-volatile semiconductor memory; hard disk or the like.

The storage 23 stores various programs (including after-mentioned moving image output control processing) executed by the controller 21, parameters necessary for executing the programs, and the like.

The second storage 24 is configured by including a non-volatile semiconductor memory, hard disk or the like.

The second storage 24 stores multiple pieces of image data.

The second storage 24 according to the present embodiment stores the multiple, pieces of image data in the form of database.

The second storage 24 may be integrated with the storage 23.

### 2-2. Configuration Example 2

As shown in FIG. 4, the output control device 3A included in the system 100A according to the above configuration example 2 includes a controller 31 (hardware processor), a communication unit 32, a storage 33, a display 34, and an operation unit 35.

The controller 31, the communication unit 32, and the storage 33 are configured similarly to the controller 21, the communication unit 22, and the storage 23 of the above output control device 2.

The display 34 is configured similarly to the monitor 3 included in the system 100 according to the above configuration example 1.

The operation unit 35 is configured similarly to the input device 4 included in the system 100 according to the above configuration example 1.

### 2-3. Operation

The controller 21 of the output control device 2 (controller 31 of the output control device 3A) configured as described above has following functions.

### Image Obtaining Function

For example, the controller 21 (controller 31) has the function of obtaining one or more pieces of image data from the modality 1.

When the medical image is a still image, the controller 21 (controller 31) obtains one piece of image data.

On the other hand, when the medical image is a moving image, the controller 21 (controller 31) obtains multiple medical images. That is, when the medical image is a moving image, the multiple medical images are multiple frame images forming the moving image.

The controller 21 (controller 31) according to the present embodiment receives the image data from the modality 1 (PACS 2A) via the communication unit 22 (communication unit 32).

When the output control device 2, 3A includes a reader of a recording medium not shown in the drawings, the controller 21 (controller 31) may read the image data from the recording medium, not via the communication unit 22.

The controller 21 (controller 31) forms an obtaining unit by having such an image obtaining function.

### Various Screen Display Functions

The controller 21 (controller 31) has a function of causing the monitor 3 (display 34) to display various screens.

The various screens include a viewer screen S or the like as shown in FIG. 5 in addition to the test list screen, for example.

The test list screen is a screen for selecting a test that involves image capturing which has been performed in the past (once or more) when the doctor performs diagnosis.

The test list screen according to the present embodiment is displayed in response to a trigger of turning on of the output control device 2, 3A, activation of the application, and a predetermined operation performed to the input device 4 (operation unit 35), for example.

A list of information (for example, test name, test date and time, and the like) indicating each test which has been performed in the past is displayed on the test list screen according to the present embodiment. The test is associated with at least one medical image.

The viewer screen S is a screen for interpreting a medical image I when the doctor performs diagnosis.

The viewer screen S according to the present embodiment is displayed when a predetermined operation (click or touch) is performed to information indicating any test in the list of information indicating tests displayed on the test list screen.

As shown in FIG. 5, the viewer screen S according to the present embodiment includes an operation button display region R₁, a test information display region R₂, an image display region R₃, and an analysis result display region R₄.

In the operation button display region R₁, there are displayed operation buttons for instructing various operations (switching of display screen, how to display medical image in the image display region R₃, and the like). When a predetermined operation (clicking, touching, or the like) is made to any of the operation buttons, the controller 21 (controller 31) performs the corresponding display switching.

In the test information display region R₂, the date and time when the test (image capturing) was performed, image capturing site, image capturing direction and the like are displayed.

In the image display region R₃, the medical image I (moving image or still image) corresponding to the image signal (image data) received from the modality 1 (PACS 2A) is displayed.

In the analysis result display region R₄, various analysis results of the medical image I displayed in the image display region R₃ are displayed.

### Information Attaching Function

The controller 21 (controller 31) has a function of attaching supplementary information to at least one of the multiple medical images I.

To be specific, when a predetermined operation (positioning the cursor and clicking, touching with a finger, or the like) is made by the user to a region desired by the user (region interesting the user) in the medical image I displayed in the image display region R₃, the controller 21 (controller 31) attaches supplementary information indicating that the region is the region of interest (ROI) for the doctor, to the medical image I.

The controller 21 and the input device 4 (controller 31 and operation unit 35) form an input unit by having such an information attaching function.

### Supplementary Information Display Function

The controller 21 (controller 31) is able to display at least part of the supplementary information in the image display region R₃.

The controller 21 (controller 31) according to the present embodiment displays the supplementary information in the image display region R₃.

The controller 21 (controller 31) according to the present embodiment displays the supplementary information in the form of overlays or annotations, for example.

The overlay O is a character string indicating information which is written to the header portion of the DICOM file in the supplementary information.

The overlays O according to the present embodiment are displayed in the region (for example, four corners of the medical image I) that overlaps, as little as possible, with the region showing the diagnosis target site.

When the medical image I is a moving image, the overlays O according to the present embodiment are displayed on all the frame images forming the moving image. That is, when the moving image is replayed, the overlays O continue to be displayed in the image display region R₃ from start to end of the replaying.

The annotation is a figure or the like indicating a feature of the medical image I in the supplementary information, for example.

The annotation according to the present embodiment includes automatic drawing annotations A₁ which are drawn by the modality 1 automatically attaching the supplementary information, and a manual drawing annotation A₂ which is drawn by the user manually attaching the supplementary information.

The automatic drawing annotations A₁ includes annotations that trace the movement of the diagnosis target site (lines indicating the positions of upper and lower ends of the lung field), for example.

When the medical image I is a moving image, the controller 21 (controller 31) displays the automatic drawing annotations A₁ on all the frame images forming the moving image. That is, when the moving image is replayed, the automatic drawing annotations A₁ continues to be displayed in the image display region R₃ from start to end of the replaying of moving image.

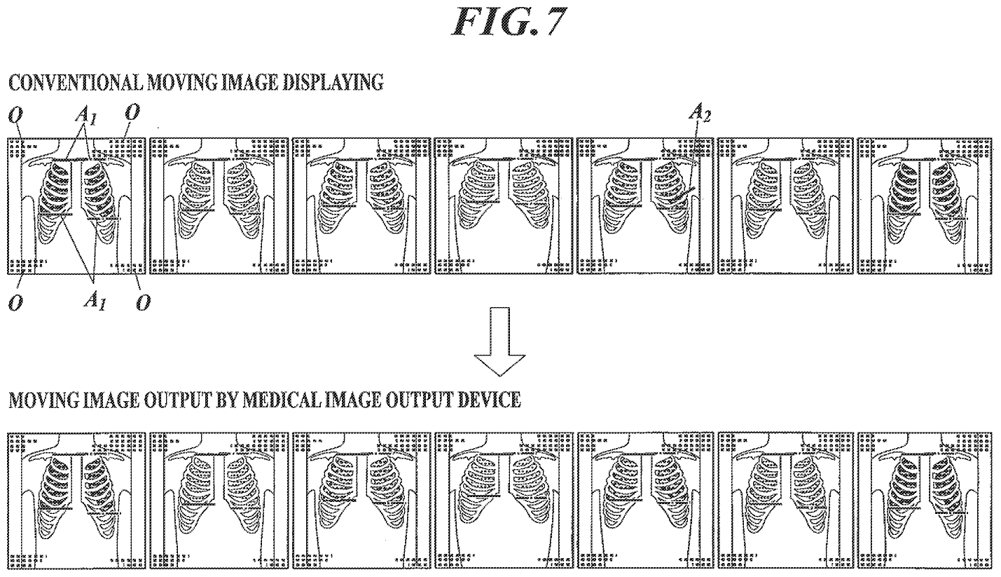

While the moving image is replayed, the controller 21 (controller 31) changes the positions of automatic drawing annotations A₁ following the movement of the diagnosis target site (see FIG. 7).

The manual drawing annotation A₂ includes an annotation marking the region selected by the user, that is, the region highly interesting the user (for example, the arrow indicating the region showing the site suspected of a legion).

When the medical image I is a moving image, the controller 21 (controller 31) displays the manual drawing annotation A₂ on only the frame image(s) which was displayed in the image display region R₃ when the user performed a predetermined operation for attaching the supplementary information.

On the other hand, when the medical image I is a still image, or when a single medical image I (frame image) is output as a still image, the controller 21 (controller 31) displays the supplementary information attached to the single medical image I in the still image.

### Information Selecting Function

The controller 21 (controller 31) has a function of selecting arbitrary supplementary information from among the supplementary information attached to the medical image I by the above information attaching function (input unit).

The controller 21 (controller 31) according to the present embodiment selects the supplementary information corresponding to a predetermined operation performed by the user (for example, supplementary information which is checked).

The controller 21 and the input device 4 (controller 31 and the operation unit 35) form a selecting unit by having such an information selecting function.

### Moving Image Outputting Function

The controller 21 (controller 31) has a function of executing the moving image output control processing as shown in FIG. 6 in response to a trigger that a predetermined condition is satisfied.

This “predetermined condition” includes, for example, that the instruction to replay the moving image is performed in a state in which the above viewer screen is displayed, that the test involving the moving image capturing is selected on the test list screen, and the like.

In this moving image output control processing, the controller 21 (controller 31) first executes first determination processing (step S1).

In the first determination processing, the controller 21 (controller 31) determines whether or not the moving image to be output is an analysis image.

Whether or not the moving image to be output is an analysis image can be determined by using various techniques which have been conventionally used. For example, the determination is made on the basis of the AE title, station name indicating the image analysis device attached to the image data, or the like, for example.

In this first determination processing, if the controller 21 (controller 31) determines that the moving image is not an analysis image (step S1: NO), the controller 21 (controller 31) executes output processing (step S4), and ends the moving image output control processing.

On the other hand, in the first determination processing, if the controller 21 (controller 31) determines that the moving image is an analysis image (step S1: YES), the controller 21 (controller 31) executes second determination processing (step S2).

In this second determination processing, the controller 21 (controller 31) determines whether or not there is supplementary information that is a target of non-displaying (target that is not to be displayed) among the supplementary information attached to the moving image (multiple medical images I) to be output.

This “non-displaying (of supplementary information)” may indicate not displaying (not displaying together with the moving image) the supplementary information (overlays O, annotations A₁, A₂) when the moving image is displayed with the supplementary information remaining in the image data of the moving image, or may indicate not displaying (not displaying together with the moving image) the supplementary information (overlays O, annotations A₁, A₂) when the moving image is displayed by deleting the supplementary information from the image data of the moving image.

The supplementary information attached to the medical image achieves an important role for diagnosis in many cases. Thus, if the supplementary information is deleted, the supplementary information is not displayed at the time of diagnosis, which possibly makes it difficult for the doctor to perform appropriate diagnosis. That is, it is basically desirable to maintain the supplementary information without deleting the supplementary information.

By controlling not to display the supplementary information when a moving image is displayed while maintaining the supplementary information in the image data of the moving image, it is possible to avoid flickering of the supplementary information when the moving image is replayed even When the supplementary information attached to the medical image forming the moving image is not deleted.

The supplementary information that is a target not to be displayed includes at least one of the following pieces of supplementary information:

supplementary information attached to a part of frame images

supplementary information attached to consecutive frame images the number of which having the supplementary information is five or less

supplementary information which was attached by the input unit

supplementary information which was selected by the selecting unit

As mentioned above, the supplementary information attached to the moving image by the modality 1 according to the present embodiment is attached to all the frame images forming the moving image. That is, in the present embodiment, the supplementary information attached to a part of the frame images is the supplementary information which was attached by the input unit.

When the presence/absence of supplementary information that is a target not to be displayed is determined on the basis of the number of consecutive frame images to which the supplementary information is attached, the number of consecutive frame images is preferably ten or less.

In the second determination processing, if the controller 21 (controller 31) determines that there is no supplementary information that is a target not to be displayed in the moving image, the controller 21 (controller 31) executes output processing (step S4), and ends the moving image output control processing.

On the other hand, in the second determination processing, if the controller 21 (controller 31) determines that there is supplementary information that is a target not to be displayed in the moving image, the controller 21 (controller 31) executes non-displaying processing (step S3).

In this non-displaying processing, the controller 21 (controller 31) controls not to display the supplementary information that is a target not to be displayed among the supplementary information attached to the multiple medical images I.

To be specific, while maintaining the supplementary information in the image data of the moving image, the controller 21 (controller 31) controls not to display the supplementary information together with the moving image during execution of after-mentioned output processing (when the image signal or the moving image file is generated), or sets the image data not to display the supplementary information together with the moving image before execution of the output processing.

While or after the control is performed not to display the supplementary information, the controller 21 (controller 31) executes the output processing (step S4).

In the case of output control device 2 according to the configuration example 1, the controller 21 transmits the image signal for replaying the moving image to the monitor 3 in this output processing. The monitor 3 which received the image signal displays the moving image.

On the other hand, in the case of output control device 3A according to the configuration example 2, the controller 31 causes the display 34 to display the moving image for which the controller 31 controlled not to display the supplementary information attached to the medical image I.

The controller 21 (controller 31) forms a control unit by executing the moving image output control processing which has been described above. As shown in FIG. 7, the controller 21 (controller 31) controls not to display the manual drawing annotation A₂ of the supplementary information attached to the medical image I at the time of displaying the moving image based on the multiple medical images I a part of which has the supplementary information attached thereto (here, the supplementary information has the manual drawing annotation A₂ displayed).

When it is obvious that the transmission source of the moving image transmits only the analysis image, the controller 21 (controller 31) may start from the second determination processing without executing the first determination processing in the moving image output control processing.

In the output processing, the controller 21 (controller 31) may generate the moving image file (for example, generalized file of MPEG4 format) for which the controller 21 (controller 31) controlled not to display the supplementary information attached to medical image I, and transmit the generated file to any other device.

In the non-displaying processing, after setting (such as raising a flag) not to display the supplementary information, the controller 21 (controller 31) may transmit the image data for which the control was performed not to display the supplementary information to any other device that performs display control of the moving image or generates the moving image file.

### 3. EFFECT

The above-described output control device 2, 3A and the system 100, 100A including this output control device 2, 3A perform controlling not to display the supplementary information attached to medical image I when displaying the moving image based on the multiple medical images I (frame images) (not to display the supplementary information together with the moving image when displaying the moving image while maintaining the supplementary information in the image data of the moving image).

Thus, according to the output control device 2, 3A and the system 100, 100A including this output control device 2, 3A, it is possible to avoid flickering of supplementary information when the moving image is replayed.

The output control device 2, 3A and the system 100, 100A perform controlling not to display the supplementary information attached to a part of the frame images (which is the supplementary information that was attached by the input unit in the present embodiment) in the above non-displaying processing. Thus, it is possible to avoid flickering of annotation at the time of replaying of the moving image.

The doctor or the like possibly requests not to display the annotation indicating the ROI among the supplementary information manually attached by the user, but requests to maintain displaying of the annotation indicating the pulmonary function (bloodstream function or ventilation function, to be more specific, bloodstream or pulmonary flow), for example.

Thus, the output control device 2, 3A and the system 100, 100A perform controlling not to display the supplementary information selected by the selecting unit among the supplementary information input by the input unit in the above non-displaying processing. Thus, it is possible to meat such user's needs.

In the above non-displaying processing, the output control device 2, 3A and the system 100, 100A perform controlling not to display supplementary information when outputting multiple medical images I including a single medical image I to which the supplementary information is attached as a moving image. Thus, it is possible to avoid flickering of information of the annotation while maintaining supplementary information (annotation) important for the diagnosis when replaying the dynamic image.

The doctor or the like possibly requests to maintain the displaying of supplementary information attached by the modality 1 (for example, annotation indicating that image processing such as BS (Bone Suppression) processing and TS (Temporal Subtraction) processing was performed) among the supplementary information attached to the medical image I.

Thus, in the above non-displaying processing, the output control device 2, 3A and the system 100, 100A does not perform the controlling not to display the common supplementary information attached to all the medical images (multiple pieces of supplementary information attached by the modality 1 in the present embodiment). Thus, it is possible to maintain displaying of the important supplementary information to be displayed while controlling not to display the supplementary information which causes flickering among the supplementary information.

### 4. OTHERS

The present invention is not limited to the above embodiment and the like, and modifications can be appropriately made within the scope of the present invention.

For example, the above system 100, 100A outputs multiple medical images I as a moving image. However, the above system 100, 100A can also be used when multiple consecutive photographs of faults are consecutively displayed.

The above description discloses, as an example of controlling not to display the supplementary information, an example of controlling not to display the supplementary information together with the moving image during execution of the output processing, or setting the image data not to display the supplementary information together with the moving image before execution of the output processing, while maintaining the supplementary information in the image data of the moving image. However, the controlling not to display the supplementary information when the moving image is displayed may be performed by deleting the supplementary information in the image data of the moving image.

The above description discloses an example of using a hard disk, semiconductor nonvolatile memory, or the like as a computer readable medium of a program according to the present invention. However, the medium is not limited to this example. As other computer readable medium, a portable recording medium such as a CD-ROM can be applied. A carrier wave can also be applied as a medium providing the program data according to the present invention via a communication line.

Although embodiments of the present invention have been described and illustrated in detail, the disclosed embodiments are made for purposes of illustration and example only and not limitation. The scope of the present invention should be interpreted by terms of the appended claims.

What is claimed is:

1. A medical image output control device that outputs multiple medical images, the device comprising a hardware processor that controls not to display supplementary information attached to a medical image among the medical images in displaying of a moving image based on the medical images.

2. The medical image output control device according to claim 1, wherein
the medical images are multiple frame images that form the moving image, and
the hardware processor controls not to display the supplementary information attached to a part of the frame images among supplementary information attached to the frame images.

3. The medical image output control device according to claim 2, wherein the hardware processor controls not to display supplementary information attached to consecutive frame images among the frame images, a number of the consecutive frame images being ten or less.

4. The medical image output control device according to claim 1, wherein
the hardware processor attaches supplementary information to at least one of the medical images, and
the hardware processor controls not to display the supplementary information that is attached by the hardware processor among the supplementary information attached to the medical image.

5. The medical image output control device according to claim 1, wherein the hardware processor does not perform controlling not to display common supplementary information that is attached to all the medical images.

6. The medical image output control device according to claim 1, wherein
the hardware processor obtains the medical images from a modality that generates the medical images and attaches supplementary information to at least a part of the medical images, and
the hardware processor does not perform controlling not to display multiple pieces of the supplementary information attached by the modality among supplementary information attached to the obtained medical images.

7. The medical image output control device according to claim 4, wherein
the hardware processor selects supplementary information from among the supplementary information that is attached to the at least one of the medical images by the hardware processor, and
the hardware processor controls not to display the supplementary information that is selected among the supplementary information that is input by the hardware processor.

8. The medical image output control device according to claim 1, wherein
the hardware processor controls to display the moving image for which the hardware processor controls not to display the supplementary information attached to the medical image among the medical images, or
the hardware processor generates a moving image file for which the hardware processor controls not to display the supplementary information attached to the medical image among the medical images, and transmits the generated moving image file to another device.

9. The medical image output control device according to claim 1, wherein
in outputting a single medical image among the medical images as a still image, the hardware processor controls to display, in the still image, supplementary information that is attached to the single medical image, and
in outputting the medical images that include the single medical image as the moving image, the hardware processor controls not to display the supplementary information.

10. A non-transitory recording medium storing a computer readable program that causes a computer of a medical image output control device which outputs multiple medical images to perform moving image output control processing of controlling not to display supplementary information attached to a medical image among the medical images in displaying of a moving image based on the medical images.

11. The recording medium according to claim 10, wherein
the medical images are multiple frame images that form the moving image, and
the program causes the computer to control not to display the supplementary information attached to a part of the frame images among supplementary information attached to the frame images in the moving image output control processing.

12. The recording medium according to claim 11, Wherein the program causes the computer to control not to display supplementary information attached to consecutive frame images among the frame images, a number of the consecutive frame images being ten or less, in the moving image output control processing.

13. The recording medium according to claim 10, wherein the program causes the computer to perform:
input processing of attaching supplementary information to at least one of the medical images, and
controlling not to display the supplementary information that is attached in the input processing among the supplementary information attached to the medical image, in the moving image output control processing.

14. The recording medium according to claim 10, wherein the program causes the computer to perform processing of not controlling not to display common supplementary information that is attached to all the medical images, in the moving image output control processing.

15. The recording medium according to claim 10, wherein the program causes the computer to perform:
obtaining processing of obtaining the medical images from a modality that generates the medical images and attaches supplementary information to at least a part of the medical images, and
processing of not controlling not to display multiple pieces of the supplementary information attached by the modality among supplementary information attached to the medical images obtained in the obtaining processing, in the moving image output control processing.

16. The recording medium according to claim 13, wherein the program causes the computer to perform:
selecting processing of selecting supplementary information from among the supplementary information that is attached to the at least one of the medical images in the input processing, and
controlling not to display the supplementary information that is selected in the selecting processing among the supplementary information that is input in the input processing, in the moving image output control processing.

17. The recording medium according to claim 10, wherein the program causes the computer to perform:
controlling to display the moving image for which controlling is performed not to display the supplementary information attached to the medical image among the medical images, or
generating a moving image file for which controlling is performed not to display the supplementary information attached to the medical image among the medical images, and transmitting the generated moving image file to another device,
in the moving image output control processing.

18. The recording medium according to claim 10, wherein the program causes the computer to perform:
controlling to display, in a still image, supplementary information that is attached to a single medical image, in outputting the single medical image among the medical images as the still image, and
controlling not to display the supplementary information, in outputting the medical images that include the single medical image as the moving image.

19. A medical image display system that displays multiple medical images, the system comprising:
an image generator that generates the medical images;
a display that displays a moving image based on the medical images; and
a hardware processor that controls not to display supplementary information attached to a medical image among the medical images in displaying of the moving image by the display.

20. A medical image display method using a medical image output control device that displays multiple medical images, the method comprising controlling, by a hardware processor included in the medical image output control device, not to display supplementary information attached to a medical image among the medical images in displaying of a moving image based on the medical images.
